(19) United States
(12) Patent Application Publication
(10) Pub. No.: US 2011/0263961 A1
(43) Pub. Date: Oct. 27, 2011

(54) Antenna for Investigating Structure of Human or Animal

(75) Inventors: Ian James Craddock, Bristol (GB); Maciej Bartlomiej Klemm, Harminster (GB); David Rhys Gibbins, Bristol (GB)

(73) Assignee: MICRIMA Limited, Bristol (GB)

(21) Appl. No.: 12/741,372

(22) PCT Filed: Nov. 4, 2008
(86) PCT No.: PCT/GB2008/003718

§ 371 Date: Aug. 23, 2010

(30) Foreign Application Priority Data
Nov. 5, 2007 (GB) 0721693.0

(51) Int. Cl. A61B 5/05

(52) U.S. Cl. 600/407

(57) ABSTRACT

A system for investigating the internal structure of a human or animal body. The system comprises one or more antennas for transmitting and/or receiving electromagnetic energy into or from the body, each antenna comprising a slot formed in a conductive element, the slot having an external boundary defined by a substantially closed internal edge of the conductive element. A receiver is configured to detect the effect of the body on the passage of the electromagnetic energy by recording one or more signals. A processor is configured to process the signal(s) in order to generate an output indicative of the internal structure of the body. The antenna slot is a continuous slot with no internal boundary, the boundary of the slot being completely defined by the internal edge of the conductive element. Each antenna may further comprise two or more conductive feed lines, each feed line coupling energy to and/or from a respective part of the slot.

### FIELD OF THE INVENTION

The present invention relates to a system and method for investigating the internal structure of a human or animal body, by transmitting electromagnetic energy into the body from one or more antennas, and detecting the effect of the body on the passage of the electromagnetic energy. Each antenna comprising a slot formed in a conductive element.

### BACKGROUND OF THE INVENTION

An eccentric annular slot antenna for breast cancer detection is described in “Eccentric annular slot antenna for breast cancer detection based on the finite-difference-time-domain method” Raja, V. K. El-Shenawee, M., Wireless Communications and Applied Computational Electromagnetics, 2005. IEEE/ACES International Conference, Publication Date: 3-7 Apr. 2005, page(s): 401-404, ISBN: 0-7803-9068-7.

The antenna comprises an annular slot having an external boundary defined by a circular internal edge of a conductive sheet, and an internal boundary defined by an “island” of material offset from the centre of the external boundary.

This antenna suffers from a number of problems. Firstly, the slot has a relatively complex geometry, requiring the “island” of material to be mounted in a precise position. Secondly, the circular internal edge must be relatively large to physically accommodate the “island”, and as a result the dimensions of the antenna (in the plane of the sheet) must be relatively large. The circular external boundary of the slot is 3 cm across and the sheet of material is approximately 5 cm across. Thirdly, the bandwidth of the slot at −10 dB is relatively low (“almost 3.87 GHz”).

Another antenna for breast cancer detection is described in AN ULTRA WIDEBAND MICROWAVE IMAGING SYSTEM FOR BREAST CANCER DETECTION, Wee Chang Khor and Marek E. Bialkowski, 10th Australian Symposium on Antennas, Sydney, Australia, 14-15 Feb. 2007.

In this case the antenna is a tapered slot antenna operating between 3.1 GHz and 10 GHz. The antenna is even larger than the antenna described in Raja et al—in this case approximately 6 cm across.

Another antenna for breast cancer detection is described in “Dielectric-filled slotline bowtie antenna for breast cancer detection” by Shannon, C. J.; Fear, E. C.; Okoniewski, M., Electronics Letters, Volume 41, Issue 7, 31 Mar. 2005 Page(s): 388-390, this is again a tapered slot measuring many cm in size.

A wide-slot antenna is described in Jia-Yi Sze, and Kin-Lu Wong, “Bandwidth enhancement of a microstrip-line-fed printed wide-slot antenna,” IEEE Transactions on Antennas and Propagation, Volume 49, July 2001 pp: 1020-1024 (referred to below as “Sze et al”). The antenna is a square, wide slot antenna with a dual fork-shaped, microstrip feed built on a substrate with relative permittivity of 4.4. It is not designed for breast cancer detection, but is instead intended to radiate into free-space with an operating bandwidth of 4.5 GHz from 2-6.5 GHz.

An object of the invention is to provide an antenna which is suitable for investigating the internal structure of a human or animal body, whilst being small and having a high bandwidth.

### SUMMARY OF THE INVENTION

A first aspect of the invention provides a system for investigating the internal structure of a human or animal body, the system comprising:
one or more antennas for transmitting and/or receiving electromagnetic energy into or from the body, each antenna comprising a slot formed in a conductive element, the slot having an external boundary defined by a substantially closed internal edge of the conductive element;
a receiver configured to detect the effect of the body on the passage of the electromagnetic energy by recording one or more signals; and
a processor configured to process the signal(s) in order to generate an output indicative of the internal structure of the body,
   characterized in that the antenna slot is a continuous slot with no internal boundary, the boundary of the slot being completely defined by the internal edge of the conductive element.

In contrast with the slot in Raja et al. which has an internal boundary, the slot is a continuous slot with no internal boundary. In contrast with the antenna in Khor et al., and the antenna in Shannon et al the slot has an external boundary defined by a substantially closed internal edge of the conductive element. Note that the boundary need not be completely closed, that is it may be substantially closed but with a small opening.

Typically each antenna further comprises one or more conductive elements for coupling energy to and/or from the slot. The conductive element may be for example a patch, a feed line, or two or more conductive feed lines, each feed line coupling with a respective part of the slot.

A second aspect of the invention provides a system for investigating the internal structure of a human or animal body, the system comprising:
one or more antennas for transmitting electromagnetic energy into the body, each antenna comprising a slot formed in a conductive element;
a receiver configured to detect the effect of the body on the passage of the electromagnetic energy by recording one or more signals; and
a processor configured to process the signal(s) in order to generate an output indicative of the internal structure of the body,
   characterized in that each antenna further comprises two or more conductive feed lines, each feed line coupling energy to and/or from a respective part of the slot.

The use of two or more conductive feed lines provides a relatively high bandwidth. In this case the slot may comprise an open slot, an annular slot, or a continuous slot with no internal boundary, the external boundary of the slot being completely defined by the internal edge of the conductive element.

The following comments apply to both aspects of the invention.

Typically each antenna is configured to transmit and/or receive radiation in a range of wavelengths including a minimum, maximum and centre wavelength, and the slot of each antenna has a maximum dimension which is smaller than the centre wavelength.

Typically the slot of each antenna has a maximum dimension which is smaller than 30 mm, and preferably smaller than 18 mm.

The system may comprise only a single antenna which is used both to transmit electromagnetic energy into the body, and also acts as part of the receiver to detect the effect of the body on the passage of the electromagnetic energy. However more preferably the system comprises two or more antennas. In this case some of the antennas may be dedicated transmitters and others may be dedicated receivers, or alternatively the antennas may act either sequentially or simultaneously as a transmitter and a receiver. In a preferred mode of operation the antennas are driven sequentially, and the remaining (non-transmitting) antennas act as receivers.

The slot may be of any shape. For instance the slot may be square (as in Sze et al) or it may have an elongated shape (for instance rectangular or oval).

The slot may be planar or non-planar. For instance, the antenna may be conformed to a surface of a human or animal body.

The conducting element in which the slot is formed may comprise a simple thin sheet of a conducting material or alternatively it may comprise a layer of conducting material on a printed circuit board or suitable microwave substrate.

A third aspect of the invention provides a system for investigating the internal structure of a human or animal body, the system comprising:
one or more antennas each antenna comprising a conductive element for transmitting and/or receiving electromagnetic energy into or from the body, and one or more slots for coupling energy to and/or from the conductive element;
a receiver configured to detect the effect of the body on the passage of the electromagnetic energy by recording one or more signals; and
a processor configured to process the signal(s) in order to generate an output indicative of the internal structure of the body.

The third aspect of the invention provides a system similar to the first and second aspects, but in which the antenna has a complementary structure.

A fourth aspect of the invention provides a method of investigating the internal structure of a human or animal body with the system of the first, second or third aspect, the method comprising:
transmitting and/or receiving electromagnetic energy into and/or from the body with the antenna(s);
detecting the effect of the body on the passage of the electromagnetic energy by recording one or more signals; and
processing the signal(s) in order to generate an output indicative of the internal structure of the body.

Preferably the method is used to investigate the internal structure of a human breast.

### BRIEF DESCRIPTION OF THE DRAWINGS

Embodiments of the invention will now be described with reference to the accompanying drawings, in which:

FIG. 1A is a plan view of a first antenna;

FIG. 1B is a side view of the antenna of FIG. 1A;

FIG. 1C is a cross-sectional side view of a variant of the antenna of FIG. 1A;

FIG. 2 is a plan view of a second antenna;

FIG. 3 is a graph showing simulated s₁₁ for slots with 17 mm x-dimension and varying z-dimension;

FIG. 4 is a graph showing simulated bore sight transfer functions at 35 mm into the phantom for the antenna configurations with varying ground plane sizes;

FIG. 5 is a graph showing simulated s11 for antennas with a 17×12 mm slot, 1×5 mm ground plane and various feed gaps; and

FIG. 6 is a schematic view of a system for investigating the internal structure of a human breast.

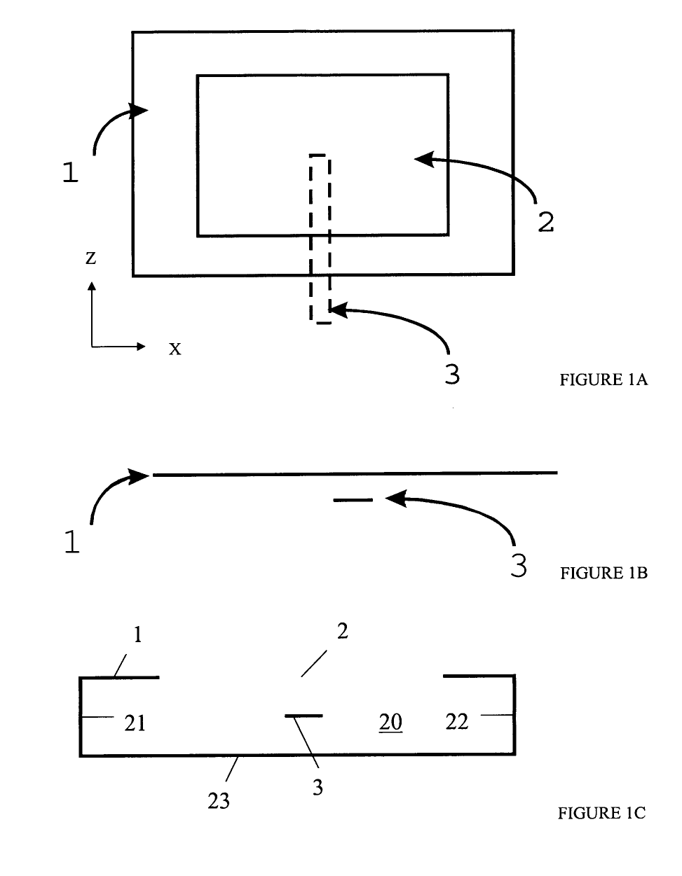

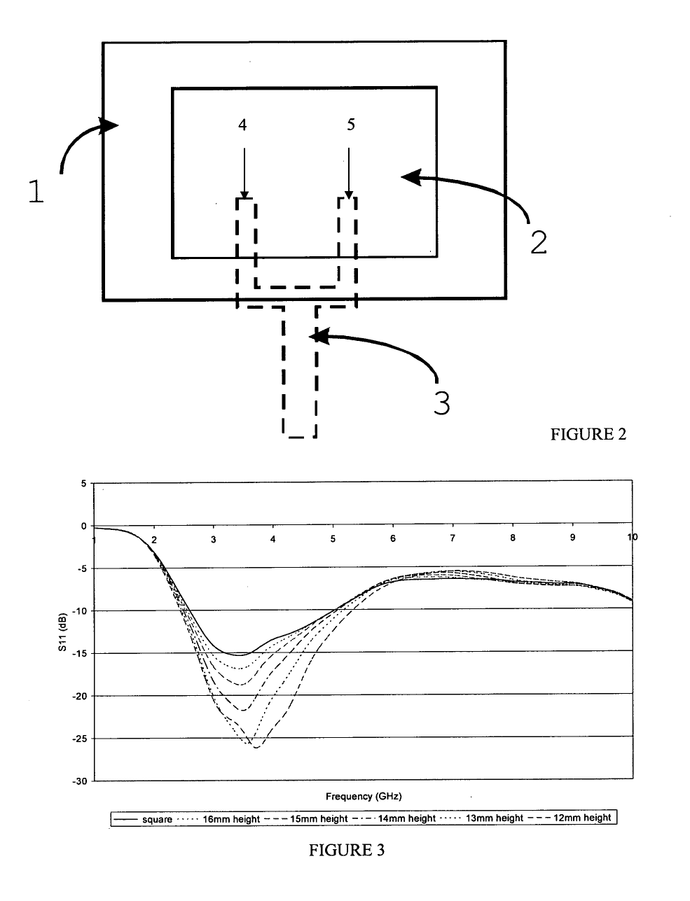

The antenna shown in FIGS. 1 and 2 comprises a slot 2 formed in a conductive element 1, the slot 2 having a rectangular external boundary defined by a substantially closed internal edge of the conductive element 1. The slot 2 is a continuous slot with no internal boundary, the boundary of the slot being completely defined by the internal edge of the conductive element 1.

A microstrip feed line 3 is spaced from the conductive element 1 as can be seen in FIG. 1B, and the distal end of the line 3 is positioned at the geometric centre of the slot 2 as can be seen in FIG. 1A. The feed line couples energy to and/or from the slot 2 in a known fashion.

The feed line 3 and conductive element 1 are mounted on opposite sides of a dielectric substrate (not shown).

In the variant shown in FIG. 1c, the rear side of the slot 2 is substantially enclosed by walls 21,22,23 of a conducting material, which together define a cavity 20. In this case the slot 2 will only radiate into a half space in front of the slot (that is, away from the cavity 20).

The antenna shown in FIG. 2 is similar to the antenna of FIGS. 1A and 1B, and the same reference numerals are used to indicate identical components. The microstrip feed line 3 splits into two parallel feed lines 4,5 each coupling energy to and/or from a respective part of the slot 2. The feed lines 4,5 are placed symmetrically with respect to the centerline of the slot. The rear side of the slot 2 in the embodiment of FIG. 2 may also be substantially enclosed by walls of a conducting material as shown in FIG. 1C.

The antennas shown in FIGS. 1 and 2 are configured to transmit radiation in a −10 dB band of wavelengths including a minimum wavelength of 9.5 mm, a maximum wavelength of 32 mm and a centre wavelength of 21 mm, and the slot 2 has a maximum dimension (that is, the diagonal) which is smaller than the centre wavelength.

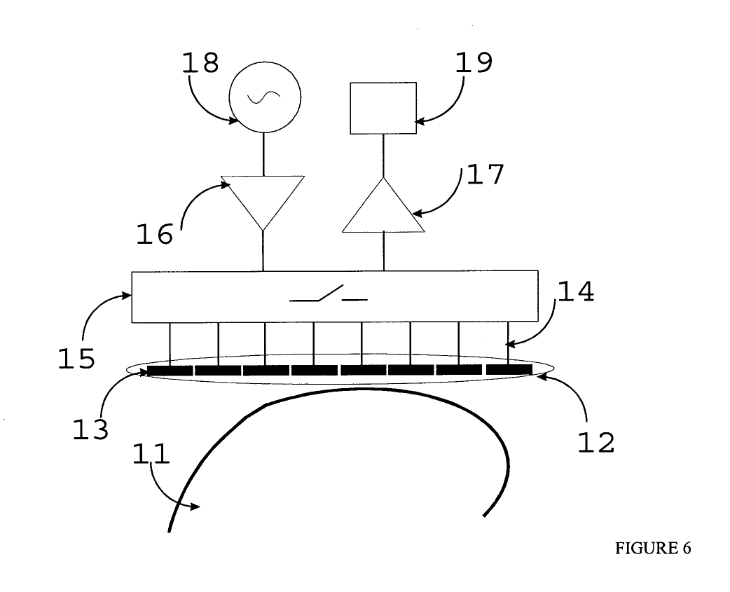

The antennas of FIGS. 1 and 2 are used in a real aperture synthetically organised radar system for breast cancer detection, shown in FIG. 6. The system operates by employing a two-dimensional array 12 of N antennas (e.g. 13) close to, or in contact with, the breast 11. Each antenna in turn transmits a pulse and the received signal at each of the other antennas is input to circuitry 19. The circuitry 19 incorporates a receiver configured to detect the effect of the body on the passage of the electromagnetic energy by recording the received signals, and a processor configured to process the signals in order to generate an output indicative of the internal structure of the breast.

The pulse generator 18 and the circuitry 19 may be time-shared, by means of a switching matrix 15 as shown in FIG. 6, as may any transmit or receive path amplification (16, 17).

The mode of operation of the system of FIG. 6 is operated as described in further detail in WO 2006/085052 A2, the contents of which is incorporated herein by reference.

The breast 11 has a relative permittivity of approximately 9. In order to minimise the size of the antenna the dielectric substrate chosen for the new antenna is RT/Duroid with a relative permittivity of ∈ᵣ=10.2. As the wavelength (λ) in the RT/Duroid substrate is smaller than that in the design of Sze et al., the dimensions of the slot and feed must be altered accordingly if the performance characteristics are to be kept. As the slot is to be in direct contact with the breast 11 and so no air dielectric interface exists, the ratio between the size of the antenna in Sze et al., and the size of the new antenna is the square root of 4.4/10.2 which is approximately 0.5. This allows the size of the slot 2 and feed fork (3,4,5) to be reduced by a factor of two using the RT/Duroid substrate. The width of the microstrip fork feed was recalculated for the new substrate permittivity. The resulting design is a square slot of 17×17 mm.

The antenna is desired to be as compact as possible and as a result the effect of reducing the size of the slot is a major consideration. The effect that altering the z-dimension of the slot (the z axis being parallel to the feed lines 3,4,5) has on the performance of the antenna can be seen in FIG. 3. FIG. 3 shows that reducing the z-dimension of the slot has little effect on the −10 dB bandwidth of the antenna although the return loss in the major null is improved by 10 dB by decreasing the slot size. This is beneficial to the antenna design and allows significant size reduction of the slot down to 17×12 mm (that is, 17 mm in the x-direction and 12 mm in the z direction).

It was found that the slot x-dimension was almost directly related to the antenna's lower bandwidth cut-off point. As the cut-off frequency is at a reasonable point no further investigation needs to be carried out.

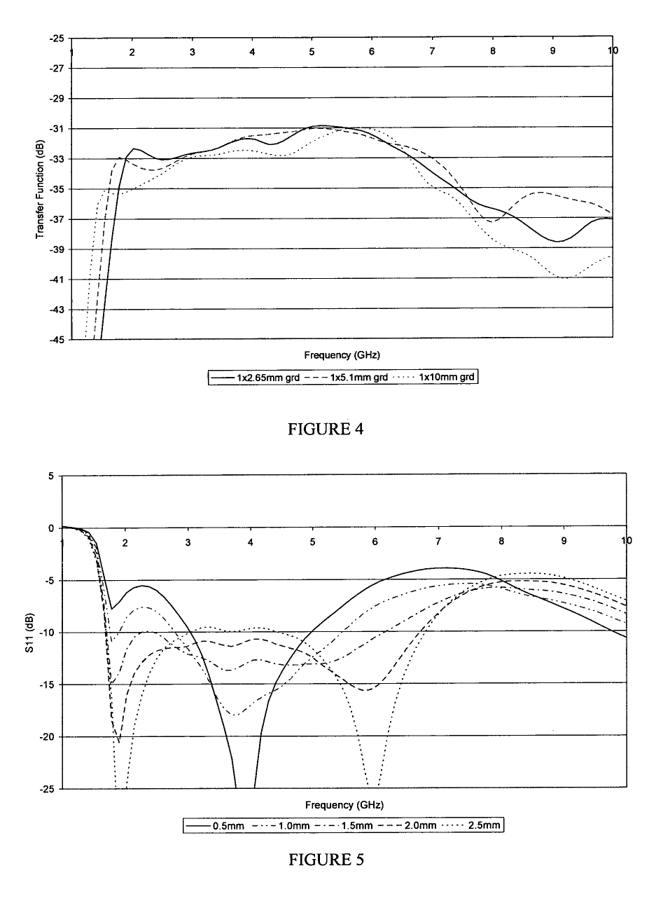

FIG. 4 shows the bore sight transfer function of various antenna designs. These plots show that regardless of the geometry of the conductive element 1 (the “ground plane”) the bore sight transfer function varies less than 10 dB in the worst case (with the conductive element 1 being 2 mm larger than the slot in the x direction, and 20 mm larger in the z direction) and 6 dB in the best case (the elements being 2 mm larger in x and 10 mm larger in z). The lack of variation and “flatness” of the transfer function across the frequency range of interest means that any signal transmitted by the antenna is likely to have minimal distortion. At angles away from bore sight the flattest transfer function is seen with a 5.1 mm ground above and below the slot. A very small or large ground causes a greater variation in the transfer function away from bore sight. The most appropriate ground size will be 1 mm in the x-direction each side of the slot and 5 mm in the z direction on each of the top and bottom of the slot.

Varying the dimension of the conductive element 1 (the ground plane) in the x-direction has very little effect on the s11 of the antenna. The general trend in the transfer function data is that there is an improvement in the antenna's performance when the size of the ground plane in the x-direction is reduced. This is beneficial in minimising the size of the antenna and allows a 1 mm ground each side of the slot to be used.

FIG. 5 shows the variation in s11 with the size of the gap between the lower edge of the slot and the fork feed (the feed gap) for the optimised antenna configuration (17×12 mm slot with a 1×5.1 mm ground). This graph shows that as the feed gap increases in size from 0.5 mm to 2.0 mm the −10 dB bandwidth increases from 2 GHz (3-5 GHz) to 5.5 GHz (1.5-7 GHz). Increasing the feed gap to values above 2 mm results in no significant increase in −10 dB bandwidth, while the return loss increases in the 2.5 GHz-0.5 GHz frequency range, to above the −10 dB level.

The transmission characteristics show a general improvement with increasing feed gap especially at higher frequencies. From these results the most effective feed gap will be 2 mm. These results also highlight the large effect that even a small change in this parameter has over the performance of the antenna. As such much care must be taken when deciding the manufacturing tolerances of such an antenna.

Although the invention has been described above with reference to one or more preferred embodiments, it will be appreciated that various changes or modifications may be made without departing from the scope of the invention as defined in the appended claims.

In particular it is noted that further miniaturisation of the antenna is possible, and prototype designs with overall dimensions less than 10 mm×10 mm have been successfully evaluated.

Also, the antenna may be replaced with a complementary structure having similar properties according to Babinet's principle. That is, the slot 2 may be replaced by a complementary patch of conductive material, and the microstrip feed lines 3,4,5 replaced by a complementary slot in a sheet of conductive material.

1. A system for investigating the internal structure of a human or animal body, the system comprising:
one or more antennas for transmitting and/or receiving electromagnetic energy into or from the body, each antenna comprising a slot formed in a conductive element, the slot having an external boundary defined by a substantially closed internal edge of the conductive element;
a receiver configured to detect the effect of the body on the passage of the electromagnetic energy by recording one or more signals; and
a processor configured to process the signal(s) in order to generate an output indicative of the internal structure of the body,
characterized in that the antenna slot is a continuous slot with no internal boundary, the boundary of the slot being completely defined by the internal edge of the conductive element.

2. The system of claim 1 wherein each antenna further comprises one or more conductive elements for coupling energy to and/or from the slot.

3. The system of claim 2 wherein the conductive elements comprise two or more conductive feed lines, each feed line coupling with a respective part of the slot.

4. A system for investigating the internal structure of a human or animal body, the system comprising:
one or more antennas for transmitting and/or receiving electromagnetic energy into or from the body, each antenna comprising a slot formed in a conductive element;
a receiver configured to detect the effect of the body on the passage of the electromagnetic energy by recording one or more signals; and
a processor configured to process the signal(s) in order to generate an output indicative of the internal structure of the body,
characterized in that each antenna further comprises two or more conductive feed lines, each feed line coupling energy to and/or from a respective part of the slot.

5. The system of claim 1 wherein one side of the slot is substantially enclosed by walls of a conducting material.

6. The system of claim 1 wherein each antenna is configured to transmit and/or receive radiation in a range of wavelengths including a minimum, maximum and centre wavelength, and wherein the slot of each antenna has a maximum dimension which is smaller than the centre wavelength.

7. The system of claim 1 herein the slot of each antenna has a maximum dimension which is smaller than 18 mm.

8. The system of claim 1, comprising two or more antennas.

9. The system of claim 1, further comprising means for sequentially driving the antennas.

10. The system of claim 1 wherein the slot has an elongated shape.

11. A system for investigating the internal structure of a human or animal body, the system comprising:
one or more antennas, each antenna comprising a conductive element for transmitting and/or receiving electromagnetic energy into or from the body, and one or more slots for coupling energy to and/or from the conductive element;
a receiver configured to detect the effect of the body on the passage of the electromagnetic energy by recording one or more signals; and
a processor configured to process the signal(s) in order to generate an output indicative of the internal structure of the body.

12. A method of investigating the internal structure of a human or animal body with the system of any preceding claim, the method comprising:
transmitting and/or receiving electromagnetic energy into and/or from the body with the antenna(s);
detecting the effect of the body on the passage of the electromagnetic energy by recording one or more signals; and
processing the signal(s) in order to generate an output indicative of the internal structure of the body.

13. The system of claim 4 wherein one side of the slot is substantially enclosed by walls of a conducting material.

14. The system of claim 4 wherein each antenna is configured to transmit and/or receive radiation in a range of wavelengths including a minimum, maximum and centre wavelength, and wherein the slot of each antenna has a maximum dimension which is smaller than the centre wavelength.

15. The system of claim 4 wherein the slot of each antenna has a maximum dimension which is smaller than 18 mm.

16. The system of claim 4, comprising two or more antennas.

17. The system of claim 4, further comprising means for sequentially driving the antennas.

18. The system of claim 4 wherein the slot has an elongated shape.
